(19) United States
(12) Patent Application Publication
(10) Pub. No.: US 2010/0280568 A1
(43) Pub. Date: Nov. 4, 2010

(54) Implantable High Efficiency Energy Transfer Module With Near-Field Inductive Coupling

(76) Inventors: Cherik Bulkes, Sussex, WI (US); Stephen Denker, Mequon, WI (US)

Correspondence Address: QUARLES & BRADY LLP, 411 E. WISCONSIN AVENUE, SUITE 2040, MILWAUKEE, WI, 53202-4497, US

(21) Appl. No.: 12/770,900

(22) Filed: Apr. 30, 2010

Related U.S. Application Data
(60) Provisional application No. 61/174,169, filed on Apr. 30, 2009.

(51) Int. Cl. A61N 1/378

(52) U.S. Cl. 607/33

(57) ABSTRACT

An apparatus includes a medical device for implantation in a blood vessel and a power supply adapted to be located outside the blood vessel. The extravascular power supply has a power transmitter that produces a radio frequency signal which is applied to an energy transmitting antenna. The energy transmitting antenna comprises first and second coils connected is series and wound around separate spaced apart, parallel axes axis wherein magnetic fields generated by each coil add together to produce a cumulative field. The receiving antenna, for positioning in a near field region of the cumulative field, has least one coil wound around a third axis that is aligned with the cumulative field.

### CROSS-REFERENCE TO RELATED APPLICATIONS

This application claims benefit of U.S. Provisional Patent Application No. 61/174,169 filed on Apr. 30, 2009.

### STATEMENT REGARDING FEDERALLY SPONSORED RESEARCH OR DEVELOPMENT

Not Applicable

### BACKGROUND OF THE INVENTION

1. Field of the Invention

The present invention relates to implantable medical devices which perform various functions in an animal, and more particularly to the wireless transfer of energy from a power source to the implantable medical device.

2. Description of the Related Art

A remedy for people with slowed or disrupted natural heart activity is to implant a cardiac pacing device which is a small electronic apparatus that stimulates the heart to beat at regular rates.

Typically the pacing device is implanted in the patient's chest and has sensor electrodes that detect electrical impulses associated with in the heart contractions. These sensed impulses are analyzed to determine when abnormal cardiac activity occurs, in which event a pulse generator is triggered to produce electrical pulses. Wires carry these electrical pulses to electrodes placed adjacent specific cardiac muscles, which when electrically stimulated contract the heart chambers. It is important that the stimulation electrodes be properly located to produce contraction of the heart chambers.

Modern cardiac pacing devices vary the stimulation to adapt the heart rate to the patient's level of activity, thereby mimicking the heart's natural activity. The pulse generator modifies that rate by tracking the activity of the sinus node of the heart or by responding to other sensor signals that indicate body motion or respiration rate.

U.S. Pat. No. 6,445,953 describes a cardiac pacemaker that for electrically stimulating tissue of an animal, comprising a generator which produces a stimulation signal having pulses occurring at a rate corresponding to a rate at which stimulation is desired. Where the stimulation controls the animal's heart rate, the stimulation signal pulses occur at the heart rate that is desired for the animal. The stimulation signal is fed to a transmitter which emits a radio frequency (RF) signal. An electrode-stent is implanted into a blood vessel of the animal at a location where the stimulation is desired, such as a blood vessel in a muscle of the heart. Upon receipt of the radio frequency signal the electrode-stent applies an electric current through tissue of the animal. In a preferred embodiment, the electrode-stent includes an antenna for receiving the radio frequency signal and a detector tuned to the frequency of the radio frequency signal. When the radio frequency signal is received, the detector produces an electric current that is applied to electrodes which in turn are in contact with the tissue to be stimulated. The use of a radio frequency signal eliminates the need for a hardwired connection between the source of the pacing signal and the stimulation electrodes. Therefore, a wire does not have to be permanently inserted through the vascular system of the animal. Although this cardiac pacing apparatus offered several advantages over other types of pacemakers, it required energy efficient stimulation systems and highly robust sensing to be developed.

Accordingly, there is a need to develop robust wireless energy transfer devices that can achieve afore-mentioned improved functionalities.

### SUMMARY OF THE INVENTION

An apparatus includes a medical device for implantation in a blood vessel and a power supply adapted to be located outside the blood vessel. Energy is conveyed wirelessly via a radio frequency signal from the power supply to the medical device for powering components of the device.

The extravascular power supply has a power transmitter connected to an energy transmitting antenna adapted for locating adjacent skin of an animal. The power transmitter produces a first radio frequency signal that is applied to the energy transmitting antenna. The energy transmitting antenna comprises a first coil wound around a first axis and a second coil connected is series with the first coil and wound around a first axis. The first and second axes are parallel and spaced apart so that B fields generated by each of the first coil and the second coil add together to produce a cumulative B field.

In one embodiment of the energy transmitting antenna, the first coil and the second coil are located coplanar side by side on a surface of a substrate.

The medical device is adapted for implantation into a blood vessel of the animal and comprises a receiving antenna for positioning in a near field region of the cumulative B field. The receiving antenna comprises at least one coil wound around a third axis that is aligned with the cumulative B field. The medical device has an electronic circuit coupled to the receiving antenna.

Several embodiments of receiving antenna are described. For example the receiving antenna may have cylindrical solenoid coil with a single or a double helix, a pair of saddle coils, or a birdcage coil.

### BRIEF DESCRIPTION OF DRAWINGS

FIG. 1 is a cardiac pacing system that includes an extravascular power supply and an intravascular medical device attached to a medical patient;

FIG. 2 is an isometric, cut-away view of a patient's blood vessels in which a receiving antenna, a stimulator and electrodes of the intravascular medical device have been implanted at different locations;

FIG. 3 is a block schematic diagram of the electrical circuitry for the intravascular medical device;

FIG. 4 illustrates the waveform of a radio frequency signal by which energy and data are transmitted to the intravascular medical device;

FIGS. 5A and B are waveform diagrams of the power supply signal and data respectively recovered from a radio frequency signal received by the intravascular medical device;

FIG. 6 depicts an exemplary pulse train transmitted from the intravascular medical device to convey information pertaining to the level of the power supply signal and to sensed physiological data for the medical patient;

FIG. 7 is a patch type energy transmitting antenna for the extravascular power supply;

FIG. 8 is a cutaway cross section of a patient's arm in which a helical energy receiving antenna has been implanted in a blood vessel;

FIG. 9 is a cutaway cross section of a patient's arm in which a saddle type energy receiving antenna has been implanted in a blood vessel

FIG. 10 shows details of a double helix, solenoid energy receiving antenna for implanting in a blood vessel;

FIG. 11 illustrates a single loop energy transmitting antenna coil for the extravascular power supply;

FIGS. 12A and 12B respectively show a parallel and series saddle configurations of an energy transmitting antenna;

FIG. 13 illustrates a double-crossed saddle configuration of an energy transmitting antenna,

FIG. 14 depicts single helix, solenoid energy receiving antenna;

FIG. 15 shows a birdcage configuration of the energy transmitting antenna;

FIGS. 16A and 16B show a multi-turn configuration involving parallel and series saddle an energy transmitting antenna folded into a flat structure to simply illustration; and

FIGS. 17A and 17B illustrate the folded and deployed forms of a birdcage type energy transmitting antenna.

### DETAILED DESCRIPTION OF THE INVENTION

Although the present invention is being initially described in the context of cardiac pacing by implanting an intravascular stimulator powered by energy from an RF signal, the present apparatus comprising of a highly efficient energy transfer module can be employed to stimulate simultaneously one or more other areas of the human body as shown in subsequent descriptions and examples. A portion of the energy transfer module may be implanted in a vein or artery of the heart or it may be embedded in cardiac muscle or skeletal muscle. In addition to cardiac applications, the energy transfer module can be a part of brain stimulation, for treatment of Parkinson's disease or obsessive/compulsive disorder for example. The electrical therapy based on the energy delivered may be applied to muscles, the spine, the gastro/intestinal tract, the pancreas, and the sacral nerve. The module may also be used as a part of the apparatus for GERD treatment, endotracheal stimulation, pelvic floor stimulation, treatment of obstructive airway disorder and apnea, molecular therapy delivery stimulation, chronic constipation treatment, and electrical stimulation for bone healing to name only a few clinical applications. The current invention can provide energy supply for two or more clinical purposes simultaneously as will be described later.

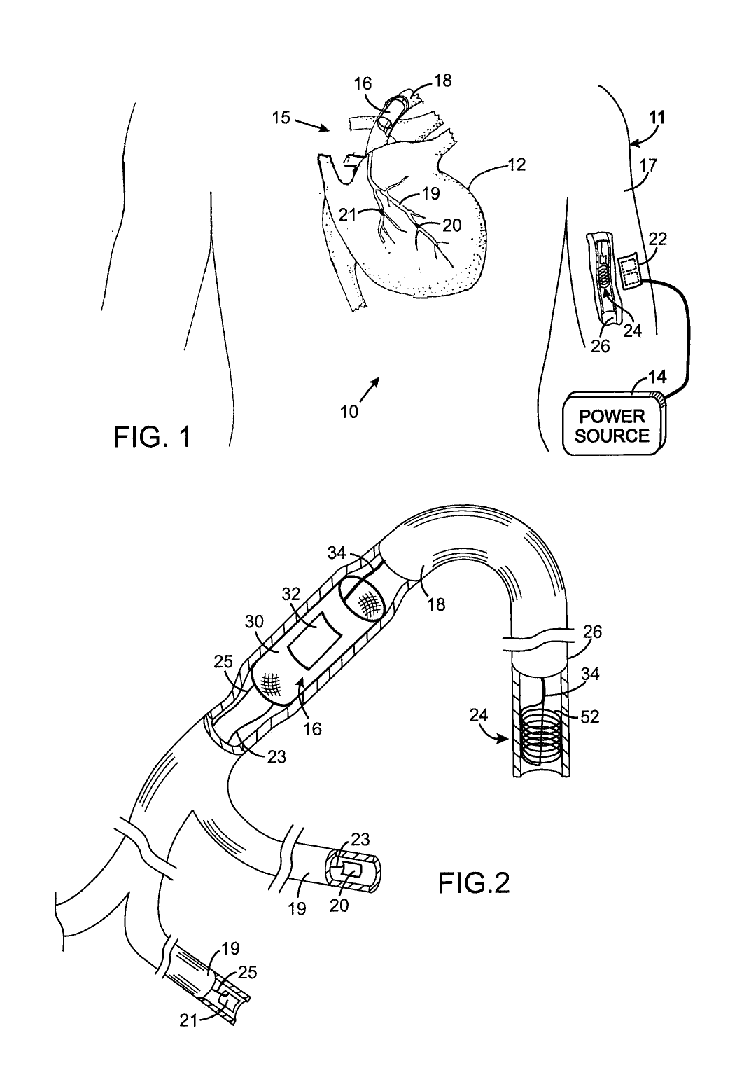

Initially referring to FIG. 1, a medical apparatus, in the form of a cardiac pacing system 10 for electrically stimulating a heart 12 to contract, comprises a power source 14, worn outside the patient's body, and a medical device 15 implanted in the circulatory system of a human patient 11. The medical device 15 receives a radio frequency (RF) signal from the extracorporeal power source 14 and its circuitry is electrically powered by the energy of that signal. Thus the power source 14 acts as a power supply for the implanted medical device 15. At appropriate times, the medical device 15 delivers an electrical stimulation pulse into the surrounding tissue of the patient thereby producing a contraction of the heart 12.

Referring to FIGS. 1 and 2, the exemplary implanted medical device 15 includes an intravascular stimulator 16 located in a vein or artery 18 in close proximity to the heart 12. The intravascular stimulator 16 has a body 30 constructed similar to a conventional expandable vascular stent. The body 30, for example, comprises a plurality of wires formed to have a memory defining a tubular shape or envelope. Those wires may be heat-treated platinum, Nitinol, a Nitinol alloy, stainless steel, plastic wires or other materials. Plastic or substantially nonmetallic wires may be loaded with a radiopaque substance which provides visibility with conventional fluoroscopy. The stimulator body 30 has a memory so that it normally assumes an expanded configuration when unconfined, but is capable of assuming a collapsed configuration when disposed and confined within a catheter assembly, as will be described. In that collapsed state, the tubular body 30 has a relatively small diameter enabling it to pass freely through the blood vasculature of a patient. After being properly positioned in the desired blood vessel, the body 30 is released from the catheter and expands to engage the blood vessel wall. The stimulator body 30 and other components of the medical device 15 are implanted in the patient's circulatory system by one or more catheters.

The body 30 has a stimulation circuit 32 mounted thereon. Electrical wires 23 and 25 extend from the stimulator 16 through the cardiac blood vasculature to locations in smaller blood vessels 19 at which stimulation of the heart is desired. At such locations, the electrical wires 23 and 25 are connected to remote electrodes 20 and 21, respectively, secured to the blood vessel wall so as to have better transfer efficiency than when if the electrode floats in the blood pool. The stimulation electrodes 20 and 21 can be embedded directly in the blood vessel wall or mounted on a collapsible body of the same type as the stimulator body 30. The electrodes 20 and 21 may be placed proximate to the sinus node (e.g. in the coronary sinus vein), the atria, or the ventricles of the heart, for example. It should be understood that additional stimulation electrodes can be provided with the stimulation circuit 32 selectively applying electrical pulses across different pairs of those electrodes to stimulate respective regions of the patient's tissue.

Because the stimulator 16 of the medical device 15 is near the heart and relatively deep in the chest of the human medical patient, an assembly 24 of transmit and receiving antennas for radio frequency signals is implanted in a blood vessel 26, i.e. a vein or an artery, in the patient's upper arm 17 or alternatively another suitable peripheral vein. The antenna assembly 24 is connected to the stimulator 16 by a cable 34. The arm blood vessel 26 is significantly closer to the skin (e.g., 6-10 mm there under) and thus antenna assembly 24 picks up a greater amount of the energy of the radio frequency signal emitted by the extracorporeal power source 14, than if the antenna assembly was located on the stimulator 16. Preferably, the power source 14 is connected to an transmitting antenna in a patch 22 or arm band on the patient's arm in close proximity to the location of the antenna assembly 24. Alternatively, another limb, neck or other area of the body with an adequately sized blood vessel close to the skin surface of the patient can be used.

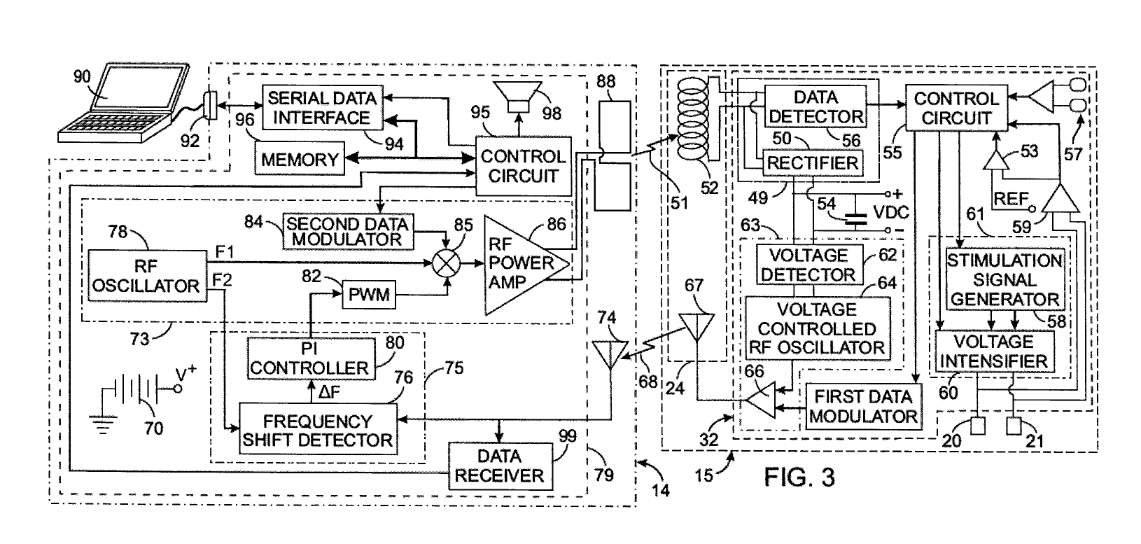

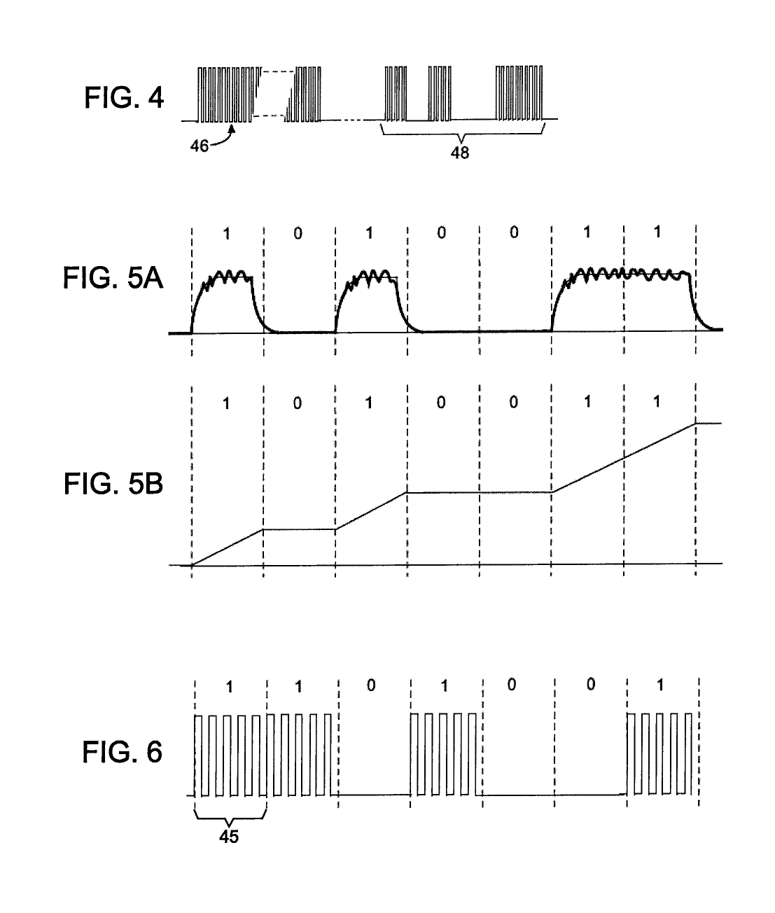

With reference to FIG. 3, the stimulation circuit 32 includes a first receiving antenna 52 within the antenna assembly 24 and that antenna is tuned to pick-up a first wireless signal 51. The first wireless signal 51 provides electrical power and carries control commands to the medical device 15. FIG. 4 depicts the format of the wireless signal 51 which comprises a periodically occurring power pulse 46 of a carrier signal at a first radio frequency (F1) that preferably is approximately 30 MHz for example to prevent excessive RF losses in the tissue of the patient. The power pulses 46 are pulse width modulated to control the amount of electrical energy conveyed to the medical device 15 and ensure that the device is sufficiently powered without wasting energy from the battery 70 in the power source 14. Alternatively, the repetition rate of the power pulses can be frequency modulated to similarly control the amount of power being conveyed.

Inside the medical device 15, the first receiving antenna 52 is coupled to a discriminator 49 that separates the signal received by the antenna into RF power and data components. A rectifier 50 in the discriminator 49 functions as a power circuit that extracts energy from the received first wireless signal. Specifically, the first wireless signal 51 is rectified to produce a DC voltage (VDC) that is applied across a storage capacitor 54 which functions as an internal power supply furnishing electrical power to the other components of the medical device. Alternatively a rechargeable battery can be used in place of the storage capacitor 54.

As necessary, the first wireless signal 51 also carries control commands that specify operational parameters of the medical device 15, such as the duration of a stimulation pulse that is applied to the electrodes 20 and 21. Those commands are sent digitally as a series of binary bits encoded on the first wireless signal 51 by fixed duration pulses 48 of the first radio frequency signal. The amplitude of the envelopes varies to modulate the control command bits on the first radio frequency signal. The discriminator 49 includes a data detector 56 that recovers data and commands carried by the first wireless signal 51. FIG. 5A illustrates the data pulse train as it appears after recovery by the data detector 56. That data detector incorporates a rectifier/capacitor circuit which suppresses the RF carrier except for the small ripple shown, however the capacitor is relatively small to have minimal affect on the data pulses except for the time constant effect on the leading and trailing edges.

The recovered data is sent to a control circuit 55 within the medical device 15, which stores the operational parameters for use in controlling a stimulation controller 61. Preferably, the control circuit 55 comprises a conventional microcomputer that has analog and digital input/output circuits and an internal memory that stores a software control program and data gathered and used by that program.

The control circuit 55 also receives data from sensor electrodes 57 that detect electrical activity of the heart and provide conventional electrocardiogram signals which are analyzed in a convention manner to determine when cardiac pacing should occur. Additional sensors for other physiological characteristics, such as temperature, blood pressure or blood flow, may be provided and connected to the control circuit 55. The control circuit stores a histogram of pacing data related to usage of the medical device and other information which can be communicated to the power source 14 or another form of a data gathering device that is external to the patient 11, as will be described.

The software executed by the control circuit analyzes the electrocardiogram signals and other physiological characteristics from the sensor electrodes 57 to determine when to stimulate the patient's heart 12. When stimulation is required, the control circuit 55 issues a command to the stimulation controller 61 which comprises a stimulation signal generator 58 that responds by applying one or more pulses of voltage from the storage capacitor 54 across various pairs of the electrodes 20 and 21 depending upon which area of the heart 12 is to be stimulated. The stimulation signal generator 58 controls the intensity and shape of the pulses. The output pulses from the stimulation signal generator 58 can be applied either directly to those electrodes 20 and 21 or via an optional voltage intensifier 60. The voltage intensifier 60 preferably is a “flying capacitor” inverter that charges and discharges in a manner that essentially doubles the power.

Determination of the voltage level, shape, and duty cycle of stimulation pulses which are applied to the electrodes 20 and 21 is made by the control circuit 55 in response to physiological characteristics detected by sensor electrodes 57. The stimulation electrodes 20 and 21 also are used for sensing to provide feedback signals for regulating the stimulation. For this purpose, the stimulation electrodes 20 and 21 are connected to inputs of a variable gain instrumentation amplifier 59 with an output that is coupled to an analog input of the control circuit 55. The output signal from the instrumentation amplifier 59 also is applied to an input of a differentiator 53 that has another input which receives a reference signal (REF). The differentiator 53 performs signal transition detection and provides an output to the control circuit 55 that indicates of time events in the sensed physiological data signal.

### Supplied Power Control

A feedback control loop is employed to regulate the electrical power supplied to the implanted medical device 15 from the power source 14. As mentioned previously, the rectifier 50 in the discriminator 49 of the medical device 15 extracts energy from the received first wireless signal 51 to charge the storage capacitor 54. FIG. 5B graphically depicts the DC voltage produced by the rectifier 50. The extracted energy charges the storage capacitor 54 that supplies electrical power to components of the implanted medical device 15. The storage capacitor 54 preferably is a supercapacitor (supercap) that is an electrochemical double layer capacitor (EDLC) hybrid between a conventional capacitor and a battery, and accordingly can be used in place of a battery to extend the life span and power capability of the storage device. However, a battery could be employed as the storage device in place of capacitor 54. In either case, the circuitry of the medical device 15 will receive is power for an extended period even if the power source 14 is not worn by the patient for short periods.

The DC voltage produced by rectifier 50 is regulated. For this function, the DC voltage is applied to a feedback transmitter 63 comprising a voltage detector 62 and a voltage controlled, first radio frequency oscillator 64. The voltage detector 62 senses and compares the DC voltage to a nominal voltage level desired for powering the medical device 15. The result of that comparison is a control voltage which indicates the relationship of the actual DC voltage derived from the received first wireless signal 51 to the nominal voltage level. The control voltage is fed to the input of the voltage controlled, first radio frequency oscillator 64 which produces an output signal at a radio frequency that varies as a function of the control voltage. For example, the first radio frequency oscillator 64 has a center, or second frequency F2, from which the actual output frequency varies in proportion to the polarity and magnitude of the control signal and thus deviation of the actual DC voltage from the nominal voltage level. For example, the first radio frequency oscillator 64 has a first frequency of 100 MHz and varies 100 kHz per volt of the control voltage deviation with the polarity of the control voltage determining whether the oscillator frequency decreases or increases from the second frequency F2. For this exemplary oscillator, if the nominal voltage level is five volts and the output of the rectifier 50 is four volts, or one volt less than nominal, the output of the voltage controlled, first radio frequency oscillator 64 is 99.900 MHz (100 MHz minus 100 kHz). That output is applied through a first RF amplifier 66 to a device transmitting antenna 67 of the implanted medical device 15, which thereby emits a second wireless signal 68.

The second wireless signal 68 also can carry data from the implanted medical device 15 to the extracorporeal power source 14. For example, physiological characteristics of the medical patient as detected by sensor electrodes 57 can be sent to the power source 14 for relaying to other equipment, such as a computer 90 in FIG. 3.

FIG. 6 depicts an example of the second wireless signal 68. That signal comprises a series of square wave pulses occur at the second radio frequency F2 which is frequency modulated to indicate the DC voltage level in the implanted medical device 15. Physiological data sensed by the medical device 15 also is carried digitally by the second wireless signal 68 as a series of binary bits. Specifically each “1” bit is encoded by a pulse 45 of several cycles of the second radio frequency for a fixed duration bit interval, and each “0” bit is encoded an absence of the radio frequency signal for the bit interval. In other words, the second wireless signal 68 is 100% amplitude modulated for a “1” bit and has zero modulation to represent a binary “0”. The space required for 100/0% amplitude modulation does not require any additional components as all that is required connector disconnect the output of the first radio frequency oscillator 64 to the device transmitting antenna 67.

To control the energy of the first wireless signal 51, the extracorporeal power source 14 contains a second receiving antenna 74, shown in FIG. 3, that picks up the second wireless signal 68 from the implanted medical device 15. Because the second wireless signal 68 indicates the level of energy received by medical device 15, this enables power source 14 to determine whether medical device requires more or less energy to be powered adequately. The second wireless signal 68 is sent from the second receiving antenna 74 to a feedback controller 75 which comprises a frequency shift detector 76 and a proportional-integral (PI) controller 80. The second wireless signal 68 is applied to the frequency shift detector 76 which also receives a reference signal at the second frequency F2 from a second radio frequency oscillator 78. The frequency shift detector 76 which acts as a receiver by comparing the frequency of the received second wireless signal 68 to the second frequency F2 and produces a deviation signal AF indicating a direction and an amount, if any, that the frequency of the second wireless signal is shifted from the second frequency F2. As described previously, the voltage controlled, first radio frequency oscillator 64, in the medical device 15, shifts the frequency of the second wireless signal 68 by an amount that indicates the voltage from rectifier 50 and thus the level of energy derived from the first wireless signal 51 for powering the implanted medical device 15.

The deviation signal AF is applied to the input of the proportional-integral controller 80 which applies a transfer function given by the expression GAIN/(1+sᵢ·τ), where the GAIN is a time independent constant gain factor of the feedback loop, T is a time coefficient in the LaPlace domain and sᵢ is the LaPlace term containing the external frequency applied to the system The output of the proportional-integral controller 80 is an error signal indicating an amount that the voltage (VDC) derived by the implanted medical device 15 from the first wireless signal 51 deviates from the nominal voltage level. That error signal corresponds to an arithmetic difference between a setpoint frequency and the product of a time independent constant gain factor, and the time integral of the deviation signal. Other types of feedback controllers may be employed.

The error signal from the feedback controller 75 is sent to the control input of a pulse width modulator (PWM) 82 within a power transmitter 73. The pulse width modulator 82 produces an output signal comprising pulses having a duty cycle that varies from 0% to 100% as dictated by the inputted error signal. The output signal from the pulse width modulator 82 is applied to an input of a second mixer 85 that also receives the first radio frequency signal at the first frequency Fl (e.g. 30 MHz) from a second radio frequency oscillator 78. The greater the duty cycle the more energy is transferred to the medical device 15. For example, a 100% duty cycle means that the first radio frequency signal is transmitted continuously and for a 25% duty cycle, the first radio frequency signal is transmitted 25% of each pulse cycle period, and off for 75% of the pulse cycle. The length of each cycle period is a function of the amount of permissible ripple in the first wireless signal 51. For example, a 100 μS cycle period is adequate for a first frequency F1 of 10 MHz. In this case, within one 100 μS cycle and 25% duty cycle, the on-time would be 25 μs containing 250 cycles of the 10 MHz signal.

Commands and data also can be sent to the implanted medical device 15 via the first wireless signal 51. An input device, such as a personal computer 90, enables a physician or other medical personnel to specify operating parameters for the implanted medical device 15. Such operating parameters are transferred to the power source 14 via a connector 92 for the input of a serial data interface 94. The digital information received by the serial data interface 94 is applied to a microcomputer based control circuit 95 and stored directly in a memory 96. At the appropriate time, the control circuit 95 formulates a message for the implanted medical device and that message is fed to a second data modulator 84 which modulates a signal with message. The output of the second data modulator 84 is fed to another input of the second mixer 85 where is combined with the pulse width modulator 82. The resultant signal is amplified by a radio frequency power amplifier 86 an applied to the energy transmitting antenna 88. The two antennas 74 and 88 for the power source 14 are contained within the patch 22 shown in FIG. 1 worn on the patient's upper arm 17. The antennas are connected to a module 79 that contains the remainder of the electronic circuitry for the power source 14. The power source 14 is powered by a battery 70, which depending upon its size, may be contained in a separate housing worn elsewhere by the patient.

### Energy Transfer

One principal aspect of the energy transfer is an implanted resonant, first receiving antenna 52 which is inductively coupled to the energy transmitting antenna 88 for the power source 14. Both those antennas have coils that are parts of separate resonant circuits tuned to the frequency of the first wireless signal 51. A resonant receiving antenna permits a higher collected energy density for a given coil volume, thus the induced voltages and currents are much higher than in a non-resonant coil. As a result, an elongated, cylindrical resonant coil with a given dimension and a high quality factor resonant circuit collect more energy from a surrounding near-field than a non-resonant coil. Antenna can be made resonant by adding a capacitor in parallel with the antenna coil to create a parallel resonant circuit, or by adding a capacitor in series to create a series resonant circuit. The apparent impedance of the resonant circuit depends on the resistive loading that may be direct or indirect. A direct load is physically connected directly across the resonant circuit. If the load is a linear resistor, it will have a dampening effect to lower the high quality factor (Q) of the resonant circuit and potentially nullify the benefit from the resonance. An indirect load can be inductively or capacitively coupled externally. The body tissue or blood pool forms an indirect load.

Another principal aspect involves taking special precautions to extract energy from the resonant circuit without excessive damping. For example, lowering the quality factor (Q) from 40 to 20 may be acceptable, however lowering the Q from 40 to less than 5 may not be acceptable. By incorporating a capacitively coupled rectifier and using the rectifier to charge a buffer capacitor, the load is only presented to the resonant circuit when the rectifier is conducting. The time constant of the buffer capacitor and the load is chosen to allow, for example, a 1% drop in voltage between charge pulses. This effectively makes the load to appear only during the top 1% of the amplitude of each input signal cycle. After initial charge-up, all that needs to be supplemented by the resonant circuit is at nearly full amplitude within the 1% mentioned in the exemplary case. The supplemented energy is provided by a power feedback as previously described.

By combining these two aspects, an efficient energy source is created. One additional aspect to consider is the transfer efficiency factor. Note that direct short wiring is the most efficient energy transfer with lowest resistance. For the wireless circuits, resonant coupled circuits are the most efficient with a high coupling factor when the primary (source) and the secondary (load) are next to each other with minimal space as in a near field scenario. In this case, the captured flux increases in a non-linear fashion. The resonant aspect focuses on a narrow band of the energy spectrum. The resonant energy has alternating electric fields coexistent with alternating magnetic fields. The energy may be derived from either one, as the fields are just a description of the two measurable aspects of the electromagnetic field transfer. However, the power dissipation in biological tissue is determined by the square of the electric field times the conductivity of the tissue divided by the density of the tissue for the computation of specific absorption rate (SAR). Therefore, the preferred energy transfer mechanism is via the magnetic field, commonly referred to as the B field. The present antennas 52 and 88 used for energy transfer are designed such that electric, or E, field is minimized. It should be noted that there are two types of electric fields: one is caused by varying magnetic field as described by Maxwell's equations, which always is present. The other types of electric field is caused by voltage sources and is minimized herein by the choice of magnetic field antennas. Hence these antennas are loops of coils that carry current and generate a magnetic field.

### Energy Transmission Antenna Configurations

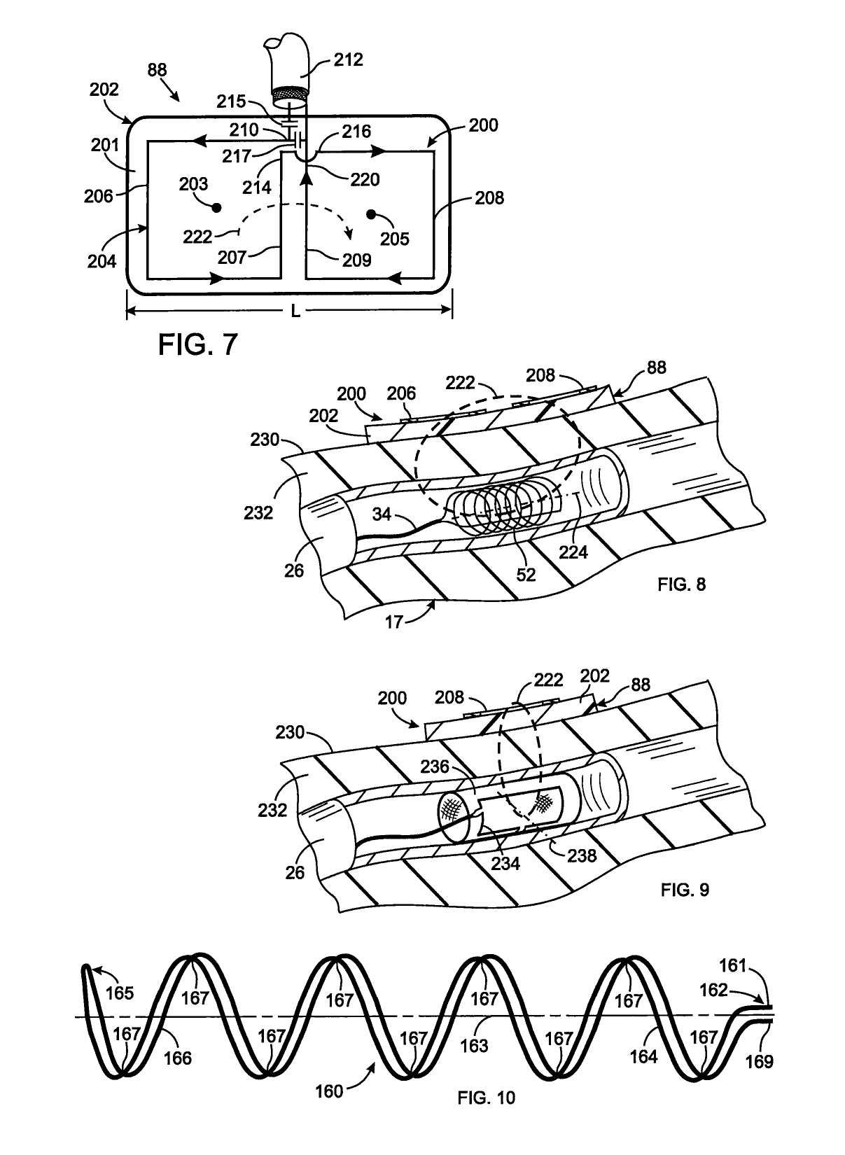

With reference to FIG. 7, the energy transmitting antenna 88 can be fabricated as a single patch antenna 200. The patch antenna 200 has a flexible, electrically insulating substrate 202 that can be attached by adhesive to the skin of the patient, such as is shown for patch 22 in FIG. 1. The substrate 202 is a sheet of material that has major surface 201 on which is formed electrically conductive pattern 204 that serves as the antenna element. That conductive pattern 204 comprises two rectangular coils 206 and 208 which are placed side by side on the major surface and which combine to form a single antenna that emits a magnetic field in a single direction. The first coil 206 extends in a single loop around a first axis 203 and the second coil 208 extends in a single loop around a second axis 205. Thus the first and second coils are coplanar and substantially flat. Each coil can be formed by a plurality of turns, or loops, on top of one another and thus may not be exactly flat, however that coil would be substantially flat. A first linear conductive section 207 of the first coil 206 is parallel to a second linear conductive section 209 of the second coil 208. A protective layer may extend over the major surface 201 covering the conductive pattern 204 and forming a laminated assembly.

The first coil 206 has a first end 210 that is connected by an impedance matching capacitor 215 to the center conductor of a coaxial cable 212 which connects to the power source 14. In some applications, an inductor is used for the impedance matching The other, or second, end 214 of the first coil 206 is connected to a third end 216 of the second coil 208 that has a fourth end 220 connected to another conductor of the coaxial cable 212. The arrows on portions of the first and second coils 206 and 208 indicate the direction of current flow through those portions and that current flow produces a magnetic (or B) field with magnetic flux. Because the two coils 206 and 208 are side by side and because of the direction of current flow through each coil, a portion of the magnetic flux produce by each coil adds cumulatively to produce and intense cumulative B field, the magnetic flux of which is indicated by curved dashed line 222. That cumulative B field curves through the interior opening of each of the rectangular coils 206 and 208 in a plane that is generally perpendicular to the plane substrate's major surface 201 (i.e., perpendicular to the plane the drawing sheet of FIG. 7). Thus the magnetic, or B, field is emitted in a direction that is orthogonal to the plane of the substrate sheet and in a direction that is parallel to the axes 203 and 205 of the two antenna coils 206 and 208.

A tuning capacitor 217 is connected across the first and fourth ends 210 and 220 of the coils and thus electrically in parallel with the antenna coil 204. The tuning capacitor 217 and the inductance and intrinsic resistance of the first and second coils 206 and 208 form a resonant circuit. That resonant circuit is tuned to the frequency Fl of the first wireless signal 51 which conveys energy between the power source 14 and the implanted medical device 15.

Although rectangular, and specifically square, coils 206 and 208 are illustrated, coils of other geometric shapes may be used. For example, each coil can have a linear side, such as side 207 or 209 in FIG. 7, with the ends of that linear side connected by an outwardly curving conductive portion. The two linear sides of such a pair of coils would be in parallel and adjacent.

As shown in FIG. 8, the energy transmitting antenna 88 is adhered to the surface 230 of the patient's skin 232 on the arm 17. This patch is placed immediately adjacent to the location of the first receiving antenna 52 which is securely implanted in a blood vessel 26 a small distance, e.g. 6-10 mm, under the surface 230 of the skin (see for example FIG. 1). Because the wavelength of the first wireless signal 51 is considerably greater than the distance between the surface of the skin and this antenna, the energy transmission utilizes near-field inductive coupling between the energy transmitting antenna 88 and the first receiving antenna 52. Looked at another way, the first receiving antenna 52 is located within the near field region of the B field produced by the energy transmitting antenna 88.

The patch antenna 200 is oriented with the length dimension L of the substrate 202 extending along the length of the arm 17 and parallel to the blood vessel 26 and thus the third axis 224 of the first receiving antenna 52. This arrangement ensures that the flux lines 222 of the B field produced by the patch antenna 200 pass longitudinally through the helical coil of the first receiving antenna 52. Optimum energy transfer is achieved when, the third axis 224 of the elongated coil of the first receiving antenna 52 is generally aligned with the flux lines from that B field. This orientation enhances the inductive coupling of the energy transmitting antenna 88 to the first receiving antenna 52.

### Receiver Coil Configurations:

A resonant coil for the first receiving antenna 52 may take various shapes and configurations based on the application and clinical purpose for the associated medical device. FIG. 10 illustrates an exemplary embodiment of a first receiving antenna 52 that comprises a coil 160 formed by an electrical conductor wound in a double helix. The coil 160 has a first terminus 161 at a first end 162 and a first helical winding 164 is wound in one rotational direction (e.g. clockwise), viewed from the first end 162, along a longitudinal axis 163 to an opposite second end 165 of the antenna coil. At the second end 165, the conductor loops into a second helical winding 166 that is wound in the same rotational direction, as viewed from the second end 165, going back to the first end 162 where the second helical winding ends at a second terminus 169. In the embodiment illustrated in FIG. 10, the first and second helical windings 164 and 166 have the same number of turns which results in every convolution of each helical winding crossing the other helical winding at two locations 167. Although the size of the coil 160 and the number of turns may differ depending upon the particular application in which the antenna is being utilized, one application for an implantable pacing device employs a coil 160 that has a diameter of five to six millimeters, a length of two inches when deployed, and twelve turns in each helical winding 164 and 166.

The cross section of the wire used to wind the double helical coil 160 is selected to provide the desired spring coefficient. A coil made from round, or circular, wire has a uniform spring coefficient whereas a ribbon (wire with a rectangular cross section) exhibits different resistances to axial versus radial deformation. Various other cross sectional shapes can be used.

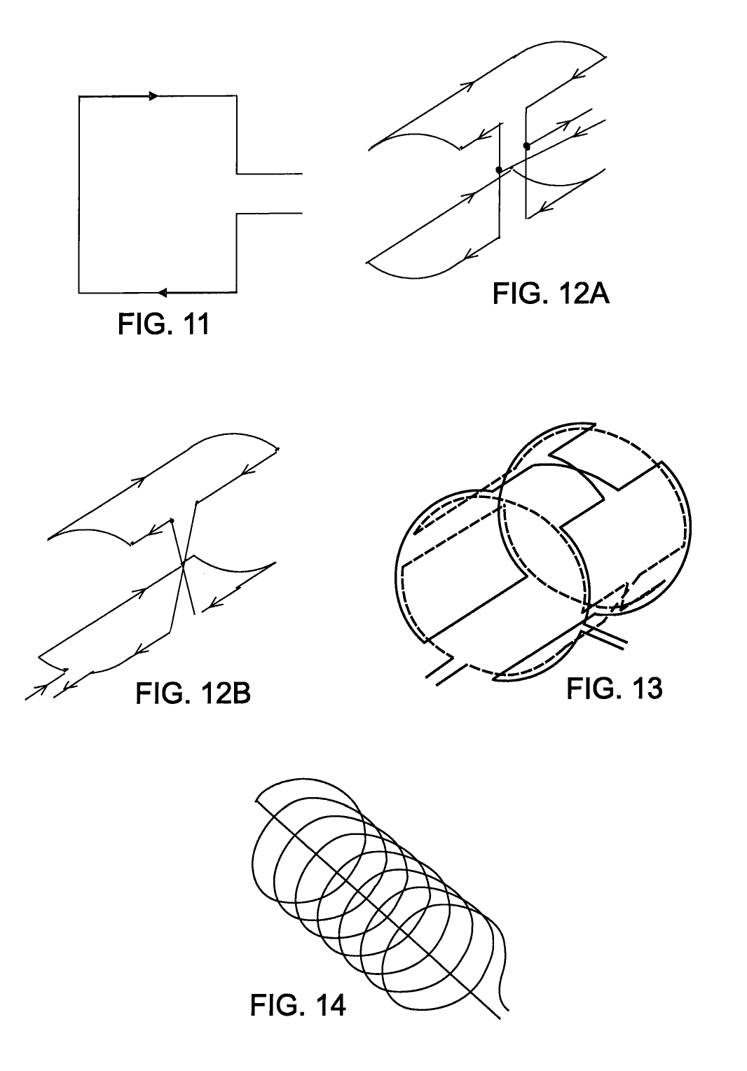

Other coils with different current paths for the first receiving antenna 52 are shown in FIGS. 11-17. FIG. 11 shows a simple loop antenna. FIGS. 12A and 12B are parallel and series saddle configurations, respectively, in which the antenna has a pair of loops curve to conform to the curvature of the blood vessel wall with the coils connected in parallel or in series. As shown in FIG. 9, these saddle coils 234 are mounted on the surface of a stent 236, which supports the first receiving antenna 52 within the blood vessel 26. With this type of a first receiving antenna, the patch antenna 200 is oriented with the substrate's length dimension L extending along the circumference of the arm 17, substantially transverse to the blood vessel 26 and thus substantially parallel to the third axis 238 about which the saddle coils are wound. This arrangement ensures that the flux lines 222 of the B field produced by the patch antenna 200 pass through both saddle coils of the first receiving antenna 52.

FIG. 13 illustrates a double-crossed saddle configuration that has two pairs of coils rotated 90 degrees with respect to each other thereby forming two crossed saddle coils. Dashed lines are used for one pair of coils to distinguish it from the other pair of coils. A 90 degree signal phase shifter (not shown) shifts the current flowing through one pair of the crossed saddle coils to be in phase with the current flowing through the other saddle coil pair so that those two currents can then be additively combined.

FIG. 14 is a solenoid configuration which has a single helical winding which is essentially one-half of the double helix in FIG. 10.

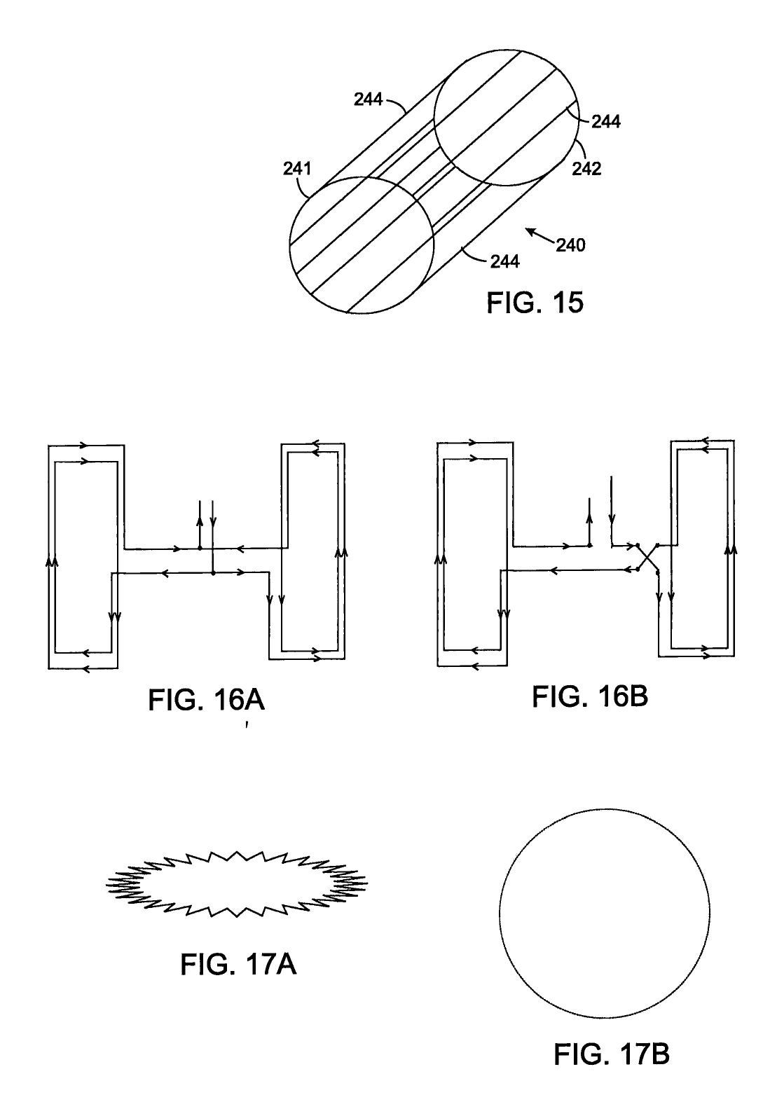

FIG. 15 shows a birdcage configuration 240 on which the first receiving antenna 52 has a pair of spaced apart end rings 241 and 242 between which a plurality of conductive rungs 244 extend. It should be noted that an omnidirectional antenna can be formed by suitable combinations of antennas mentioned above. In one variation, single coil solenoid antenna shown in FIG. 14 can be superimposed on the double crossed saddle configuration in FIG. 13 or the birdcage configuration in FIG. 15 to produce an omnidirectional antenna. In another variation, a double helix antenna shown in FIG. 10 can be superimposed on the double crossed saddle configuration or the birdcage configuration to produce an omnidirectional antenna.

Furthermore, it should be noted that the antennas shown in FIGS. 11, 12A, 12B and 13 are shown as having coils with a single loop, but such coils may comprise have multiple turns. A multiple turn configuration involving a parallel saddle is shown folded into a flat structure for illustration simplicity in FIG. 16A, in one example. In another example, a multiple turn configuration involving a series saddle arrangement is shown in FIG. 16B folded into a flat structure for illustration simplicity. As yet another example, a birdcage configuration can be constructed as having collapsible end rings shown as folded structures shown in FIG. 17A. The end rings expand to conform the regular shape when deployed as shown in FIG. 17B.

The foregoing description was primarily directed to a preferred embodiment of the invention. Although some attention was given to various alternatives within the scope of the invention, it is anticipated that one skilled in the art will likely realize additional alternatives that are now apparent from disclosure of embodiments of the invention. Accordingly, the scope of the invention should be determined from the following claims and not limited by the above disclosure.

1. A medical apparatus comprising:
an extravascular power supply having a power transmitter that produces a first radio frequency signal, and an energy transmitting antenna to which the first radio frequency signal is applied and which is adapted for locating adjacent skin of an animal, the energy transmitting antenna comprising a first coil wound around a first axis and a second coil connected is series with the first coil and wound around a first axis, wherein the first and second axes are parallel and spaced apart so that B fields generated by each of the first coil and the second coil add together to produce a cumulative B field; and
a medical device adapted for implantation into a blood vessel of the animal and comprising a receiving antenna for positioning in a near field region of the cumulative B field and comprising at least one coil wound around a third axis that is aligned with the cumulative B field, the medical device having an electronic circuit coupled to the receiving antenna.

2. The medical apparatus as recited in claim 1 wherein the energy transmitting antenna and receiving antenna both resonate at a frequency of the first radio frequency signal.

3. The medical apparatus as recited in claim 1 wherein the energy transmitting antenna further comprises a substrate sheet with a major surface and the first coil and the second coil are located side by side on the major surface.

4. The medical apparatus as recited in claim 3 wherein the first coil has a first linear conductive section and the second coil has a second linear conductive section that is adjacent to the first linear conductive section.

5. The medical apparatus as recited in claim 4 wherein the first linear conductive section is parallel to the second linear conductive section.

6. The medical apparatus as recited in claim 1 wherein the first coil and the second coil are rectangular.

7. The medical apparatus as recited in claim 1 wherein the first coil and the second coil are substantially flat and coplanar.

8. The medical apparatus as recited in claim 1 wherein the receiving antenna comprises a helical coil.

9. The medical apparatus as recited in claim 1 wherein the receiving antenna comprises a double helical coil.

10. The medical apparatus as recited in claim 1 wherein the receiving antenna comprises a saddle coil.

11. The medical apparatus as recited in claim 1 wherein the receiving antenna comprises a cylindrical birdcage coil.

12. A medical apparatus comprising:
an extravascular power supply having a power transmitter that produces a first radio frequency signal, and an energy transmitting antenna coupled to the power transmitter for receiving the first radio frequency signal and adapted for locating adjacent skin of an animal, the energy transmitting antenna comprising a first coil and a second coil connected is series and located coplanar side by side so that B fields generated by each of the first coil and the second coil additively combine to produce a cumulative B field; and
a medical device adapted for implantation into a blood vessel of the animal and comprising a receiving antenna for positioning in a near field region of the cumulative B field and comprising at least one coil wound around a third axis that is aligned with the cumulative B field, the medical device having an electronic circuit coupled to the receiving antenna.

13. The medical apparatus as recited in claim 12 wherein the energy transmitting antenna and receiving antenna both resonate at a frequency of the first radio frequency signal.

14. The medical apparatus as recited in claim 12 wherein the first coil has a first linear conductive section and the second coil has a second linear conductive section that is adjacent to the first linear conductive section.

15. The medical apparatus as recited in claim 14 wherein the first linear conductive section is parallel to the second linear conductive section.

16. The medical apparatus as recited in claim 12 wherein the first coil and the second coil are rectangular.

17. The medical apparatus as recited in claim 12 wherein the first coil and the second coil are substantially flat and coplanar.

18. The medical apparatus as recited in claim 12 wherein the receiving antenna comprises a helical coil.

19. The medical apparatus as recited in claim 12 wherein the receiving antenna comprises a double helical coil.

20. The medical apparatus as recited in claim 12 wherein the receiving antenna comprises a saddle coil.

21. The medical apparatus as recited in claim 12 wherein the receiving antenna comprises a cylindrical birdcage coil.
